(19) United States
(12) Patent Application Publication
(10) Pub. No.: US 2004/0230288 A1
(43) Pub. Date: Nov. 18, 2004

(54) Medical devices adapted for controlled in vivo structural change after implantation

(76) Inventor: Arthur L. Rosenthal, Boston, MA (US)

Correspondence Address: Paul F. Prestia, Ratner & Prestia, P.C., P.O. Box 980, One Westlake, Suite 301 (Berwyn), Valley Forge, PA, 19482, US

(21) Appl. No.: 10/124,254

(22) Filed: Apr. 17, 2002

(51) Int. Cl. A61F 2/06; A61F 2/02

(52) U.S. Cl. 623/1.13; 623/1.38; 623/1.44; 623/23.75

(57) ABSTRACT

A medical device comprising a combination of one or more dynamic structural elements and one or more non-dynamic structural elements and having a first structural form immediately after deployment in a body, the device adapted to change in vivo to a second structural form due to a change induced in the one or more dynamic structural elements without releasing debris greater than a predetermined size. An exemplary device may comprise at least one biodegradable element contained radially between a first confining member and a second confining member.

### TECHNICAL FIELD

This invention relates generally to medical implants and, more specifically, to medical implants having the ability to change shape after implantation.

### BACKGROUND OF THE INVENTION

Medical devices for placement in a human or other animal body are well known in the art. One class of medical devices comprises endoluminal devices such as stents, stent-grafts, filters, coils, occlusion baskets, valves, and the like. As is known in the art, a stent is typically an elongated device used to support an intraluminal wall. Such a stent may also have a prosthetic graft layer of fabric or covering lining the inside and/or outside thereof. Such a covered stent is commonly referred to in the art as an intraluminal prosthesis, an endoluminal or endovascular graft (EVG), or a stent-graft. Although stent-grafts may be used in any number of applications, the use of stent-grafts for repairing abdominal aortic aneurysms (AAA) is an area of particular interest. Other devices, such as filters or occlusion devices (also known as wire clusters), may have similar structures to stents and may be placed in a body lumen by similar methods. As used herein, the term “medical device” refers to any type of device that is deployed in a human or other animal body, endoluminally or otherwise. As used herein, the term “endoluminal device” refers to covered and uncovered stents, filters, wire clusters, and any other device that may be placed in a lumen. The term “stent” as used herein is a shorthand reference referring to a covered or uncovered stent. Typical mechanisms for expansion of endoluminal devices include spring elasticity, balloon expansion, and self-expansion of a thermally or stress-induced return of a memory material to a pre-conditioned expanded configuration.

Traditionally, an endoluminal device, such as a stent-graft deployed in a blood vessel at the site of a stenosis or aneurysm, is implanted endoluminally, i.e. by so-called “minimally invasive techniques” in which the device, typically restrained in a radially compressed configuration by a sheath, crocheted or knit web, or catheter, is delivered by a delivery system or “introducer” to the site where it is required. The introducer may enter the body from an access location outside the body, such as through the patient's skin (percutaneous methods), or by a “cut down” technique in which the entry blood vessel is exposed by minor surgical means. The term “proximal” as used herein refers to portions of the stent or delivery system relatively closer to the end of the delivery system extending outside of the body, whereas the term “distal” is used to refer to portions relatively farther from this outside end. When the introducer has been threaded into the body lumen to the deployment location, the introducer is manipulated to cause the endoluminal device to be ejected from the surrounding sheath or catheter in which it is restrained (or alternatively the surrounding sheath or catheter is retracted from the endoluminal device), whereupon the endoluminal device expands to a predetermined diameter at the deployment location, and the introducer is withdrawn.

These less invasive therapies for complex diseases have intensified the need for versatile medical devices that can adapt, in vivo, to the ever-changing dynamics of the morphological and physiological processes. For example, aneurysmal disease and initimal hyperpalasia are characterized by non-uniform morphology, uncontrolled cellular proliferation, malignancy, cytotoxicity, and variable tissue strength. Additionally, after the aneurysm is isolated, the structural form of the prosthesis may change in an uncontrolled and undesired manner as the aneurysm shrinks, potentially causing serious problems. Furthermore, the continuous pulsation of the aorta can cause failures due to metal fatigue. Thus, medical devices that are capable of resisting failure and that can change as the morphology of the surrounding lumen changes are desirable. In general, versatile medical devices capable of disease management on many fronts, such as drug delivery, prevention of cellular proliferation, and maintenance of structural integrity, are desired.

It is known to coat endoluminal devices on their outer surfaces with a substance such as a drug releasing agent growth factor, or the like, or to fabricate a stent comprising a hollow, perforated tubular wire through which drugs can be injected and released into the body lumen. Numerous publications and patents detail the benefits of using certain drugs to inhibit restenosis following vascular trauma, including but not limited to U.S. Pat. No. 5,616,608, No. 6,716,981, and No. 5,733,925. There is a continued need in the field, however, to improve control of the release of drugs into the body.

It is further known to provide endoluminal devices composed of biodegradable elements. As used herein, the term “biodegradable” means degradable inside a biological entity by any means of degradation, such as erosion, dissolution, biological or chemical attack, or any mechanism known in the art. A disadvantage of biodegradable devices, however, is that as the devices biodegrade, degradation products may remain in the endoluminal fluid, causing serious problems. Therefore, it is desirable to minimize the potential for biodegradable devices to create debris in the endoluminal fluid.

### SUMMARY OF THE INVENTION

It is to be understood that both the foregoing general description and the following detailed description are exemplary, but not restrictive, of the invention.

One aspect of the invention comprises a medical device comprising a combination of at least one dynamic structural element and at least one non-dynamic structural element. The device has a first structural form immediately after deployment in a body and is adapted to change in vivo to a second structural form due to a change induced in the at least one dynamic structural element without releasing debris greater than a predetermined size. The dynamic structural element, for example, may comprise a biodegradable structural element. Where the device is an endoluminal device for deploying in a lumen having a lumen wall and endoluminal fluid flow through the lumen, the biodegradable structural element may be positioned to avoid direct exposure to the endoluminal fluid flow, such as positioned in radially outward of a confining layer positioned between the biodegradable element and the endoluminal fluid flow. In another embodiment, the device may comprise a plurality of confining layers, such as graft or mesh layers, wherein the at least one biodegradable structural element is positioned radially between two of the confining layers.

In one embodiment, the device comprises a stent comprising one or more non-biodegradable filaments and one or more biodegradable sutures holding corresponding portions of the one or more non-biodegradable filaments together. In another embodiment, the device comprises a stent-graft comprising a graft having a distal end, a proximal end, and an intermediate portion. The device has a distal stent for affixing the distal end of the graft against a lumen wall and a proximal stent for affixing the proximal end of the graft against the lumen wall. The one or more biodegradable structural elements are positioned to interface with the intermediate portion of the graft.

In certain embodiments, the device may comprise a first biodegradable structural element having a first set of degradation properties and a second biodegradable structural element having a second set of degradable structural properties that is different than the first set of degradation properties. For example, the first biodegradable structural element may comprise a different material of construction and/or a different geometry than the second biodegradable structural element.

Another aspect of the invention comprises an endoluminal device comprising at least one biodegradable element contained radially between a first confining member and a second confining member. The confining members may be graft members, or any mesh member, such as a textile or wire mesh. The device may be adapted for implantation in a lumen having a wall, in which one or more ablumen-side confining members collectively may have at least one different characteristic than the one or more lumen-side confining members collectively. The different characteristics may comprise porosity; permeability to one or more agents that causes the biodegradable element to degrade or to one or more elutants, such as biologically or pharmacologically active agents generated by degradation of the biodegradable element; or receptivity to neointimal tissue formation.

In one embodiment, the device may comprise a first confining layer of ePTFE and a second confining layer of a textile fabric, in which the biodegradable element comprises a drug-encapsulated co-polymer, such as a microsphere.

Yet another aspect of the invention comprises a method of securing a biodegradable component of an endoluminal device to the endoluminal device until a desired degree of biodegradation has occurred, the method comprising securing the degradable component within a covering of one or more confining materials.

Still another aspect of the invention comprises a method of enabling an endoluminal device to vary with respect to one or more characteristics over time after implantation without releasing debris greater than a predetermined size into a stream of endoluminal fluid. The method comprises positioning the device with a first non-biodegradable structural element positioned relative to a second non-biodegradable structural element, the relative position of the first non-biodegradable structural element to the second non-biodegradable structural element controlled by one or more biodegradable structural elements. The device has at least one confining layer positioned between the biodegradable structural element and the stream of endoluminal fluid to prevent debris greater than a predetermined size to pass through the confining layer. The device is deployed in an endoluminal location, the one or more biodegradable structural elements is induced to degrade; and the relative position of the first non-biodegradable structural element to the second non-biodegradable structural element is changed in response to the degradation of the one or more biodegradable structural elements.

Yet another aspect of the invention comprises a method of treating a body lumen having a known first morphology prior to treatment and an expected, different, second morphology after treatment, the method comprising deploying an endoluminal device comprising at least one dynamic structural element adapted to undergo a predictable or controlled change after deployment without releasing debris greater than a predetermined size.

Still another aspect of the invention comprises an endoluminal prosthesis comprising one or more biodegradeable structural elements at selected locations, the biodegradeable structural elements adapted to provide the stent with initial rigidity at the selected locations and to biodegrade in vivo over a period of time to provide a consequent reduction in rigidity at the selected locations. The one or more biodegradable structural elements may be captured within one or more confining layers adapted to be retained in place upon degradation of the biodegradable element. The confining layers may have a porosity that selectively permits permeation or non-permeation by degradation products of the biodegradable structural elements, components of surrounding biological fluid, or both. The prosthesis may further comprise non-biodegradable elements and one or more graft layers adapted to retain the non-biodegradable elements in a position relative to one another following the degradation of the biodegradable elements. One specific embodiment may comprise a first non-biodegradable stent at a distal end of the prosthesis, a second non-biodegradable stent at a proximal end of the prosthesis, one or more biodegradable elements in an intermediate portion of the prosthesis between the distal and proximal stents, and a graft lining or covering extending between the distal stent and the proximal stent.

Yet another aspect of the invention comprises a medical device comprising a mesh layer comprising one or more co-extruded polymer wire filaments, each co-extruded polymer wire filament comprising a non-biodegradable core encapsulated by a biodegradable polymer. The mesh layer may comprise a radially outward layer of an endoluminal device adapted to have a first mesh size before degradation of the biodegradable polymer and a second mesh size after degradation of the biodegradable polymer, such as for promoting intimal growth after the device has been deployed and the biodegradable polymer has degraded. A radially inward confining layer may be provided to prevent debris from decomposition of the radially outward layer from entering the stream of endoluminal fluid.

### BRIEF DESCRIPTION OF THE DRAWING

The invention is best understood from the following detailed description when read in connection with the accompanying drawing, in which:

FIG. 1 shows a longitudinal slice of an exemplary embodiment of an endoluminal device of the present invention having a biodegradable element confined between a confining layer and the walls of a body lumen;

FIG. 2A shows a longitudinal slice of an exemplary endoluminal device comprising biodegradable elements sandwiched between two confining layers, before degradation of the biodegradable elements;

FIG. 2B shows the portion of the slice shown in FIG. 2A within dashed lines, after degradation of the biodegradable elements;

FIG. 3 shows a plan view of an exemplary stent cut longitudinally and laid flat, showing two types of biodegradable elements having different degradation characteristics;

FIG. 4A shows a longitudinal slice of a portion of an exemplary device having confining layers with different properties in different longitudinal portions of the device, wherein the different properties are provided by having different pairs of confining layers in each longitudinal portion;

FIG. 4B shows a longitudinal slice of a portion of an exemplary device having confining layers with different properties in different longitudinal portions of the device, wherein the different properties are provided by having an additional confining layer in one longitudinal portion;

FIG. 5 shows a longitudinal slice of a portion of an exemplary device comprising microspheres confined bewteen an ePTFE layer and a textile layer;

FIG. 6A shows a cross-sectional view of an exemplary mesh comprising co-extruded biodegradable polymer wires before degradation of the polymer layer;

FIG. 6B shows a plan view of the exemplary mesh of FIG. 6A after degradation of the polymer encapsulating layer; and

FIG. 6C shows a longitudinal slice of an exemplary endoluminal device comprising the mesh of FIG. 6A as a radially outward layer and having a radially inward confining layer.

### DETAILED DESCRIPTION OF THE INVENTION

The invention will next be illustrated with reference to the figures wherein the same numbers indicate similar elements in all figures. Such figures are intended to be illustrative rather than limiting and are included herewith to facilitate the explanation of the apparatus of the present invention.

Referring now to FIG. 1, there is shown one embodiment of the invention comprising a medical implant that is dynamically able to change with the morphology of the lumen into which it is implanted. Device 10 comprises a plurality of biodegradable structural elements 12 and a plurality of non-biodegradable structural elements 14. Biodegradable elements may be referred to generally as “dynamic” elements because they can change over time, whereas standard non-biodegradable elements may generally be referred to as non-dynamic elements because they typically do not change over time. Dynamic elements may also be provided that change over time by different mechanisms than biodegradation, however, and therefore non-dynamic elements comprise elements that do not change by any mechanism over time. In accordance with the invention, it is advantageous to prevent the biodegradable element from breaking off in large pieces into the endoluminal fluid 16. Thus, as shown in FIG. 1, device 10 may further comprise a confining layer 18, such as a graft or wire mesh, interposed between biodegradable elements 12 and endoluminal fluid 16. Device 10 may then be deployed such that the biodegradable elements are confined between the confining layer 18 and lumen wall 21, thereby preventing direct exposure of the biodegradable elements 12 to the flow of endoluminal fluid 16. Confining layer 18 is chosen to prevent debris greater than a predetermined size from passing through it. The predetermined size may be relatively large, such as in the case of a confining layer 18 comprising a wire or textile mesh, or may be relatively small, such as on a molecular level where the confining layer comprises a polymer or polymer coated fabric, for example.

In another embodiment, shown in FIGS. 2A and 2B, the biodegradable elements 24 may be sandwiched between two confining layers 18a and 18b, one on the ablumen side (18a) and one on the lumen side (18b) of the stent framework 11. In this configuration, as the biodegradable elements degrade, the debris created by the degradation cannot leave the confinements of the two grafts. Although shown in FIG. 1 with all the biodegradable elements confined between confining layer 18 and lumen wall 21 and in FIGS. 2A and 2B with all the biodegradable elements confined between confining layers 18a and 18b, devices in accordance with this invention may use some combination of these confinement techniques to confine the biodegradable elements, and various embodiments using either or both confinement mechanisms, may have all or less than all of the biodegradable elements so confined. Suitable materials of construction for the confining layer may include any materials typically used for grafts, such as but not limited to: polyester, such as DACRON® polyester, manufactured by E. I. du Pont de Nemours and Company of Wilmington, Del.; polyethyleneterepthalate (PET); polyetheretherketone (PEEK); polysulfone; polytetrafluroethylene (PTFE); expanded polytetrafluroethylene (ePTFE); fluorinated ethylene propylene (FEP); polycarbonate urethane; a polyolefin, such as polypropylene, polyethylene, or high density polyethylene (HDPE); silicone; and polyurethane. The confining layer may comprise any type of mesh, including a wire mesh, such as but not limited to a wire mesh comprising nitinol, elgiloy, stainless steel, or the like, having a mesh size (the interstitial area among the intersecting filaments of the mesh) chosen to prevent debris above the mesh size from getting into the bloodstream.

Referring now to FIGS. 6A and 6B, the biodegradable element may comprise a mesh 60 comprising non-biodgradable and biodegradable elements, such as a mesh comprising biodegradable polymer encapsulated wire 62. The biodegradable polymer encapsulated wire may be manufactured by any process known in the art, including co-extrusion, coating, or dipping processes. The biodegradable polymer encapsulated wire 62 may comprise a first biodegradable polymer encapsulating a biodegradable or non-biodegradable polymer core, or may comprise a polymer 64 encapsulating a metal wire 66, as shown in FIG. 6A. For example, the biodegradable polymer encapsulated wire may comprise PLGA co-extruded with elgiloy or nitinol. The polymer may be impregnated with a biologically or pharmacologically active substance that may be eluted as the biodegradable polymer decomposes. Any combination of polymer and metal wire or multiple polymers (two or more than two) known or practical in the art may be used. Mesh 60 comprising co-extruded biodegradable polymer wire 62 comprising metal wire 66 with a polymer 64 encapsulation provides a mesh with a first mesh area m₁ before degradation of the polymer encapsulation as shown in FIG. 6A, and a second mesh area m₂ after degradation, as shown in FIG. 6B. This allows for intimal growth over time into the mesh comprising the remaining metal wire 66. The polymer 64 used for encapsulating wire 66 may be impregnated with a substance that promotes intimal growth. Where mesh 60 is used in a radial outward layer of an endoluminal device 61, as shown in FIG. 6C, a radially inward confining layer 68 may be provided to trap any debris generated by mesh 60 as it biodegrades between layer 68 and lumen wall 21, so that no debris above a predetermined size enters the stream of endoluminal fluid 16.

In one embodiment, the biodegradable elements may be used to provide increased rigidity along the entire length of or in certain portions of an endoluminal device during introduction of the device into the body lumen, and may be designed to subsequently degrade after deployment. For example, as shown in FIG. 1, biodegradable structural elements 12 interface with an intermediate portion 19 of graft 18 to provide support and rigidity during introduction, but later degrade, leaving only non-biodegradable stent 22a at the distal end and non-biodegradable stent 22b at the proximal end. Such an embodiment may be particularly useful for repair of aneurysms, such as abdominal aortic aneurysms, where the morphology of the aneurysm tends to change over time. In such embodiments, it is advantageous to ultimately be left with distal and proximal stents used to hold a biocompatible graft in place between the stents, where the graft is free to change its structural form along with the aneurysm. The use of a biodegradable portion between the non-biodegradable stents as shown in FIG. 1, provides rigidity during deployment, making such devices easier to deploy, but then the biodegradable portion degrades, leaving the graft free to change its shape along with the body lumen. Although shown in FIG. 1 in an embodiment with only a lumen-side confining layer, the biodegradable intermediate portion may also be provided in an embodiment wherein the biodegradable portion is confined between ablumen-side and lumen-side confining layers, such as is shown in FIGS. 2A and 2B.

In other embodiments, such as is shown in FIG. 2A, the biodegradable elements 24 may comprise sutures designed to hold non-biodegradable portions 26a and 26b of stent 11 together initially in a first configuration, but to weaken and break after deployment to allow the stent to attain a second configuration (shown in FIG. 2B) that more conforms to the shape of the body lumen as the morphology of the lumen changes. Thus, for example, as shown in FIG. 2A, device 20 may have a first configuration having one or more gathered regions 28, that straighten as shown in FIG. 2B, after degradation of the biodegradable components causes the device to elongate. The change in the device is not limited to a change in length, however, and may include other changes in geometry, including but not limited to a change in diameter. Although depicted in FIGS. 2A with exaggerated gathered regions 28 to help in visualizing the impending change in length, the gathered regions may in actuality be less disruptive than depicted. In particular, gathered regions 28 in the inner layer 18b may not form a restriction of any significance, and typically not to the degree depicted in FIG. 2A. Gathered regions 28 are also depicted as being greater in the outer layer 18a than in the inner layer 18b, consistent with there being different materials of construction for each layer. It should also be recognized that, depending on the elasticity of the materials used for those layers, there may be no appreciable difference in the degree of gathering in each layer, and in some circumstances, neither layer may show significant gathering at all. In other embodiments, the same material of construction may be used for both layers, making the gathering characteristics equal.

Suitable materials for the biodegradable elements include biopolymers such as but not limited polyglycolide (PGA), polylactides (PLA) such as L-lactide (LPLA) or DL-lactide (DLPLA), poly(ε-caprolactone) (PCL), poly(dioxanone) (PDO), poly(lactide-co-glycolide) trimethylene carbonate copolymer (PGA-TMC), and poly(DL-lactide-co-glycolide) (DLPLG). As is well-known in the art, the glycolide DL-lactide copolymer DLPLG may be provided in a number of different copolymer forms (e.g. 85/15, 75/25, 65/35, 50/50 and the like), each having different material properties including different rates of degradation. The various properties of these materials are well-known and documented in the art, such as documented by John C. Middleton and Arthur J. Tipton in their article “Synthetic Biodegradable Polymers as Medical Devices,” published in Medical Plastics and Biomaterials Magazine in March 1998, incorporated herein by reference. Other materials such as hyaluronic acid, chitosan, and polysaccharides, may also be used, particularly for drug delivery, as may any naturally or synthetically derived biodegradable material known in the art, contemplated, or practical for use in medical devices.

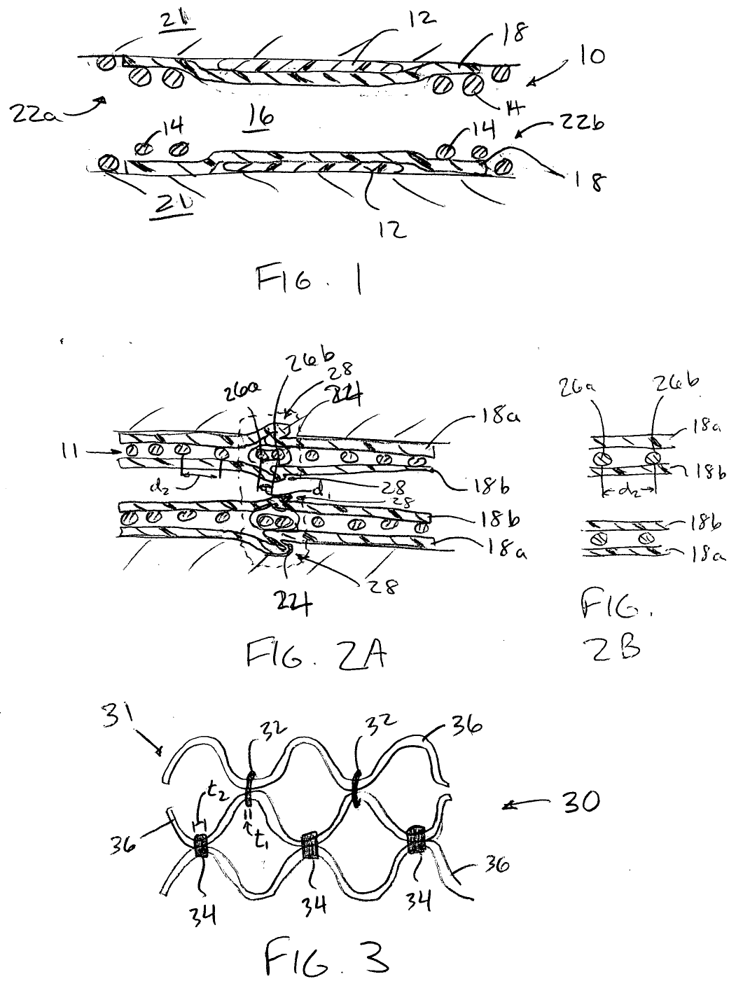

In the embodiment shown in FIG. 3, device 30 comprises a first biodegradable structural element 32, such as a suture between non-biodegradable elements 36 of a stent 31, having a first set of biodegradation properties and a second biodegradable structural element 34 having a second set of biodegradation properties that is different than the first set of biodegradation properties. For example, the first set of biodegradation properties may comprise a first exposure time after which first biodegradable structural element 32 is designed to degrade. The second set of biodegradation properties may comprise a second exposure time after which the second degradable structural element is designed to degrade that is different than the first exposure time. The difference in exposure time may be provided by using different materials of construction for the first and second biodegradable structural elements 32 and 34, respectively.

As shown in FIG. 3, the first biodegradable structural element may comprise a first geometry and the second biodegradable structural element may comprise a second geometry different from the first geometry, such as where the first biodegradable structural element has a first thickness t₁ or effective diameter and the second biodegradable structural element has a second thickness t₂ or effective diameter. The different geometry may also comprise the use of hollow, cavity, or porous portions in the biodegradable members, such as is described in U.S. Pat. No. 5,980,564 to Stinson, incorporated herein by reference. A combination of different geometry and different materials may also be used to provide different degradation properties. Although shown in FIG. 3 where the biodegradable members are sutures holding together apices of a zig-zag stent, the biodegradable elements may be any type of element, structural or non-structural, and may have any geometry or perform any function known in the art. Similarly, although shown as a stent, the device may be any type of medical device, endoluminal or otherwise. The device architecture is not limited to a zig-zag, wound or even filamentary architecture, and may comprise any architecture known in the art, including filamentary or cut-tube architectures, wound or braided architectures, and the like. For example, the stent may comprise a cut-tube architecture where the biodegradable elements comprise restraining bands over a portion of the cut-tube architecture, such as is shown in U.S. Pat. No. 6,350,277, incorporated herein by reference. In another embodiment, the biodegradable elements may comprise filaments incorporated into a woven or braided architecture, including devices comprising all biodegradable elements as well as stents comprising combinations of biodegradable and non-biodegradable elements.

As shown in FIGS. 2A and 2B, the confining members 18a and 18b may have the same characteristics or at least one different characteristic, such as porosity, permeability, or receptivity to neointimal tissue formation. The difference in permeability may be with respect to one or more agents, such as enzymes, in the surrounding endoluminal fluid that cause the biodegradable element to degrade or with respect to one or more elutants generated by degradation of the biodegradable element. For example, where one of the elutants generated by degradation of the biodegradable element is a biologically or pharmacologically active agent, it may be more desirable for that biologically or pharmacologically active agent to be directed to the lumen wall rather than discharged into the bloodstream. In such a case, the ablumen-side confining member 18a may be designed with a greater permeability with respect to that agent than the lumen-side confining member 18b. For example, an impermeable membrane, such as polyethylene (PET), may be provided on one side, and a porous membrane, such as ePTFE, on the opposite side. Membranes of various materials of construction are well known in the art to be porous, impermeable, or semipermeable to all or to particular certain substances. Even different grades of the same type of membrane may be used on opposite sides. For example, it is known in the art to provide ePTFE coated fabrics with different pore sizes adapted to allow molecules of different sizes to pass through. It is also known in the art to construct membranes having holes or pores therein of various sizes.

Drug-eluting properties may also be isotonically or enzymatically controlled, and therefore the permeability of the confining layers to enzymes that cause drug elution or to the drug itself may be used to affect the rate of drug elution. For example, a semi-permeable membrane such as an ePTFE membrane may be used to exclude or contain a large molecule from passing through the membrane. For a large molecule bound to a smaller molecule at the membrane boundary, the large molecule cannot pass through, but the small molecule may be released from the large molecule by an ionic and/or pH effect and then pass through the membrane. Exemplary large molecules bound to small molecules include but are not limited to heparin bound growth factor (HBGF), or alginate bound molecules such as are typically used in some slow-release anti-inflammatory or acetylsalicilic acid (ASA) (aspirin) preparations. It should be recognized that although only a single layer on each side of the biodegradable element is shown in the figures, a plurality of layers may be provided, with various combinations of layers and their properties being used to effect the desired characteristics.

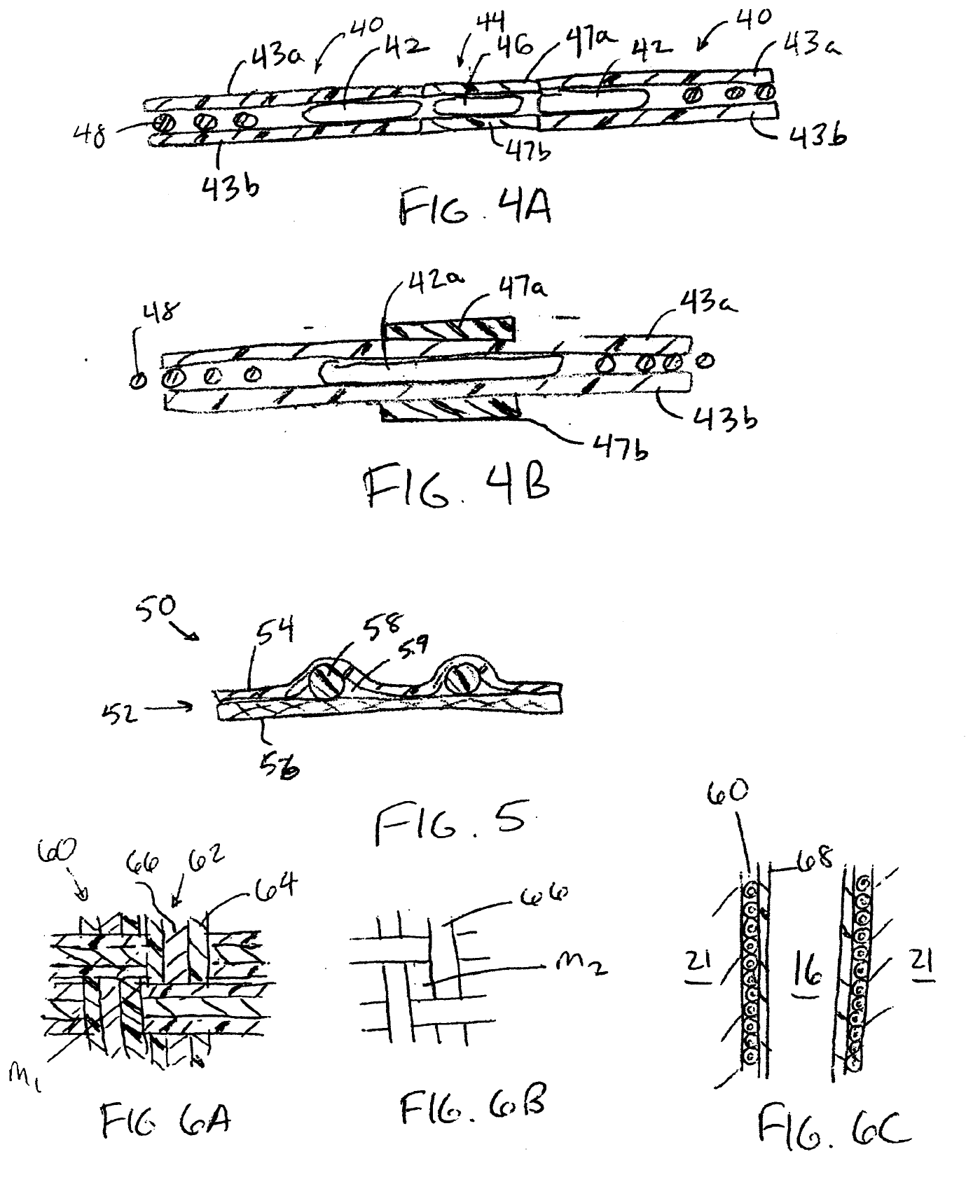

Another embodiment, shown in FIG. 4A, may have a first longitudinal portion 40 comprising at least a first degradable element 42 or portion thereof sandwiched between a first plurality of graft members 43a and 43b having a first set of one or more characteristics, and a second longitudinal portion 44 comprising a second degradable element 46 or portion thereof sandwiched between a second plurality of graft members 47a and 47b having a second set of one or more characteristics different from the first set of one or more characteristics. The first and second degradable elements may be discretely different elements 42 and 46, as shown in FIG. 4A, or may be different portions of a single element 42a, as shown in FIG. 4B. The different properties imparted by the sandwich of confining layers may be provided by having a single layer over the elements, where the single layer comprises different discrete graft members attached together in series, such as grafts 43a and 47a as shown in FIG. 4A, or may be provided by adding graft layers parallel to one another in portions where different characteristics are desired, such as is shown in FIG. 4B. In FIG. 4B, grafts 43a and 43b extend over essentially the entire length of the device, while grafts 47a and 47b are provided only in an intermediate portion where a different rate of biodegradation may be desired. Although shown in FIGS. 4A and 4B with three distinct sections: a middle section and two end sections, with the middle section having different characteristics than the two end sections, fewer or more longitudinal sections with different characteristics may be provided, and the different characteristics may be distributed in any configuration imaginable to impart a desired effect. Furthermore, in embodiments in which graft 47a has different characteristics than graft 47b and/or graft 43a has different characteristics than graft 43b, the collective characteristics of the combination of layers 43a and 47a typically has different characteristics than the collective characteristics of the combination of layers 43b and 47b. An embodiment having collectively different characteristics in the lumen-side layer as compared to the ablumen-side layer may comprise layer 47a but not layer 47b, or vice versa.

In one embodiment, shown in FIG. 5, the endoluminal device 50 may comprise a hybrid fabric 52 comprising first graft layer 54 comprising ePTFE and a second graft layer 56 comprising a textile fabric. Biodegradable components, such as drug-eluting element 58, may be contained within the interstitial spaces 59 of the hybrid fabric. The drug-eluting element may comprise a bioerodable drug-encapsulated co-polymer, such as in the shape of a microsphere, as shown in FIG. 5.

Securing biodegradable elements of an endoluminal device within a covering of one or more confining materials as described above provides a method of securing the biodegradable elements to the endoluminal device until a desired degree of biodegradation has occurred, thereby minimizing debris in the bloodstream, and the risks associated therewith, resulting form the degradation of the biodegradable elements.

The structures of the present invention also provide a method of enabling an endoluminal device to vary with respect to one or more characteristics over time after implantation, without creating debris in the bloodstream greater than a predetermined size. For example, referring to FIG. 2A, the method can be generally described as positioning a device with a first non-dynamic structural element 26a positioned relative to a second non-dynamic structural element 26b, the relative position of 26a to 26b controlled by one or more dynamic structural elements, such as biodegradable sutures 24. After the device is implanted in an endoluminal location, biodegradation induces sutures 24 to fail such that the relative position of 26a to 26b changes from distance d₁ to distance d₂. Debris in the bloodstream is prevented by enclosing biodegradable suture 24 within confining members 18a and 18b. Different sections of the device can be designed to undergo dynamic changes at different times by enclosing one biodegradable element between one set of confining members and a second biodegradable element between another set of confining members, and varying the characteristics of the confining members, by using biodegradable elements with different degradation characteristics, or a combination thereof.

Finally, it should be noted that the present invention has been described and shown as simplistically as possible to convey the general aspects of the invention. The various features of the invention may be combined with any other features known in the art for use in the medical device field. For example, although the exemplary endoluminal devices are shown herein as non-branching devices, branching devices, such as devices for repairing abdominal aortic aneurysms (AAA) may particularly benefit from incorporating the various concepts of this invention. Endoluminal devices may be used in vascular applications, including but not limited to cardiovascular applications, ureteral applications, or in any body lumen in which the use of endoluminal devices is known in the art. In addition to impregnation of biodegradable elements with biologically or pharmacologically active active agents, active agents may also be provided as coatings in particular regions of the devices in accordance with this invention, as is known in the art.

The term “biologically or pharmacologically active agent” refers to any substance, whether synthetic or natural, that has a pharmacological, chemical, or biological effect on the body or a portion thereof. Suitable biologically or pharmacologically active agents that can be used in this invention include without limitation: glucocorticoids (e.g. dexamethasone, betamethasone), antithrombotic agents such as heparin, cell growth inhibitors, hirudin, angiopeptin, aspirin, growth factors such as VEGF, antisense agents, anti-cancer agents, anti-proliferative agents, oligonucleotides, antibiotics, and, more generally, antiplatelet agents, anti-coagulant agents, antimitotic agents, antioxidants, antimetabolite agents, and anti-inflammatory agents may be used. Antiplatelet agents can include drugs such as aspirin and dipyridamole. Aspirin is classified as an analgesic, antipyretic, anti-inflammatory and antiplatelet drug. Dipyridamole is a drug similar to aspirin in that it has anti-platelet characteristics. Dipyridamole is also classified as a coronary vasodilator. Anticoagulant agents may include drugs such as heparin, protamine, hirudin and tick anticoagulant protein. Anti-cancer agents may include drugs such as taxol and its analogs or derivatives, such as paclitaxel. Taxol and its analogs or derivatives are also classified as cell-growth inhibitors. Antioxidant agents may include probucol. Anti-proliferative agents may include drugs such as amlodipine and doxazosin. Antimitotic agents and antimetabolite agents may include drugs such as methotrexate, azathioprine, vincristine, vinblastine, 5-fluorouracil, adriamycin and mutamycin. Antibiotic agents can include penicillin, cefoxitin, oxacillin, tobramycin, and gentamicin. Suitable antioxidants include probucol. Also, genes or nucleic acids, or portions thereof may be used. Such genes or nucleic acids can first be packaged in liposomes or nanoparticles. Furthermore, collagen-synthesis inhibitors, such as tranilast, may be used. Additional biologically or pharmacologically active substances and carriers for these substances for use as coatings on medical devices are listed in U.S. Pat. No. 6,364,856; No. 6,358,556; and No. 6,258,121; each of which is incorporated herein by reference.

Suitable biologically or pharmacologically active agents may be used in conjunction with any non-active carriers as are known in the art. Non-biodegradable materials used in the devices of the present invention may comprise any materials known in the art, including metals and metal alloys such as nitinol, elgiloy, and stainless steel, or non-biodegradable polymers.

Although illustrated and described herein with reference to certain specific embodiments, the present invention is nevertheless not intended to be limited to the details shown. Rather, various modifications may be made in the details within the scope and range of equivalents of the claims and without departing from the spirit of the invention.

1. A medical device comprising a combination of one or more dynamic structural elements and one or more non-dynamic structural elements and having a first structural form immediately after deployment in a body, the device adapted to change in vivo to a second structural form due to a change induced in the one or more dynamic structural elements without releasing debris greater than a predetermined size.

2. The device of claim 1, wherein at least one of the dynamic structural elements comprises a biodegradable structural element.

3. The device of claim 2, wherein the device comprises an endoluminal device adapted to be positioned in a lumen having a lumen wall and an endoluminal fluid flow through the lumen.

4. The device of claim 3, wherein the at least one biodegradable structural element is positioned to avoid direct exposure to the endoluminal fluid flow.

5. The device of claim 3 further comprising a radially inward confining layer, wherein the biodegradable structural element is positioned radially outward of the confining layer.

6. The device of claim 2, wherein the device comprises a plurality of confining layers and the at least one biodegradable structural element is positioned radially between two of the confining layers.

7. The device of claim 2, wherein the device comprises one or more confining layers and one or more biodegradable structural elements, in which all of the one or more biodegradable structural elements are either positioned radially outward of a confining layer or positioned radially between two confining layers.

8. The device of claim 2, wherein the device comprises a stent comprising one or more non-biodegradable filaments and one or more biodegradable sutures holding corresponding portions of the one or more non-biodegradable filaments together.

9. The device of claim 2, wherein the device comprises a stent-graft comprising a graft having a distal end, a proximal end, and an intermediate portion; a distal stent for affixing the distal end of the graft against a lumen wall; a proximal stent for affixing the proximal end of the graft against the lumen wall, and in which the one or more biodegradable structural elements are positioned to interface with the intermediate portion of the graft.

10. The device of claim 9, wherein the device comprises a plurality of graft layers and at least one of the biodegradable structural elements is positioned between two of the graft layers.

11. The device of claim 2 comprising a first biodegradable structural element having a first set of degradation properties and a second biodegradable structural element having a second set of degradable structural properties that is different than the first set of degradation properties.

12. The device of claim 11, wherein the first set of degradation properties comprises a first exposure time after which the first biodegradable structural element is designed to degrade and the second set of biodegradation properties comprises a second exposure time after which the second degradable structural element is designed to degrade that is different than the first exposure time.

13. The device of claim 12 in which the first biodegradable structural element comprises a different material of construction than the second biodegradable structural element.

14. The device of claim 12 in which the first biodegradable structural element comprises a first geometry and the second biodegradable structural element comprises a second geometry that is different from the first geometry.

15. The device of claim 14 wherein the first geometry comprises a first thickness and the second geometry comprises a second thickness.

16. (Cancelled)

17. The device of claim 55, wherein at least one of the first confining member and the second confining member comprises a graft member.

18. The device of claim 55, wherein at least one of the first confining member and the second confining member comprises a mesh.

19. The device of claim 18, wherein the mesh comprises a wire mesh.

20. The device of claim 19, wherein the wire mesh comprises nitinol.

21. The device of claim 18, wherein the mesh comprises a textile mesh.

22. The device of claim 55, wherein the first confining member comprises one or more radially outward confining members positioned radially outward of the biodegradable element and the second confining member comprises one or more radially inward confining members positioned radially inward of the biodegradable element, the one or more radially inward confining members having at least one collective characteristic that is different than at least one collective characteristic of the one or more radially outward confining members collectively.

23. The device of claim 22, wherein the at least one characteristic comprises porosity.

24. A medical device comprising at least one biodegradable element contained radially between a first confining member comprising one or more radially outward confining members positioned radially outward of the biodegradable element and a second confining member comprising one or more radially inward confining members positioned radially inward of the biodegradable element wherein at least one of said confining members is adapted to prevent debris from passing therethrough, the one or more radially inward confining members having at least one collective characteristic that is different than at least one collective characteristic of the one or more radially outward confining members collectively and wherein the at least one characteristic comprises permeability to one or more agents that causes the biodegradable element to degrade.

25. A medical device comprising at least one biodegradable element contained radially between a first confining member comprising one or more radially outward confining members positioned radially outward of the biodegradable element and a second confining member comprising one or more radially inward confining members positioned radially inward of the biodegradable element wherein at least one of said confining members is adapted to prevent debris from passing therethrough, the one or more radially inward confining members having at least one collective characteristic that is different than at least one collective characteristic of the one or more radially outward confining members collectively and wherein the at least one characteristic comprises receptivity to neointimal tissue formation.

26. A medical device comprising at least one biodegradable element contained radially between a first confining member comprising one or more radially outward confining members positioned radially outward of the biodegradable element and a second confining member comprising one or more radially inward confining members positioned radially inward of the biodegradable element wherein at least one of said confining members is adapted to prevent debris from passing therethrough, the one or more radially inward confining members having at least one collective characteristic that is different than at least one collective characteristic of the one or more radially outward confining members collectively and wherein the at least one characteristic comprises permeability to one or more elutants generated by degradation of the biodegradable element.

27. The device of claim 26, wherein one of the elutants comprises a biologically or pharmacologically active agent.

28. The device of claim 55, wherein the device comprises a first longitudinal portion comprising at least a first degradable element sandwiched between a first plurality of confining members having a first set of one or more characteristics, and a second longitudinal portion comprising a second degradable element sandwiched between a second plurality of confining members having a second set of one or more characteristics different from the first set of one or more characteristics.

29. The device of claim 55, wherein the device comprises a first confining layer comprising ePTFE and a second confining layer comprising a textile fabric.

30. The device of claim 29, wherein the biodegradable element comprises a drug-encapsulated co-polymer.

31. The device of claim 29, wherein the biodegradable element comprises a microsphere.

32. The device of claim 55, wherein the biodegradable element comprises a drug-encapsulated co-polymer.

33. The device of claim 55, wherein the biodegradable element comprises a microsphere.

34. The device of claim 55, wherein the biodegradable element comprises a structural element.

35. The device of claim 55, wherein the biodegradable element comprises a drug-eluting element.

36. A method of securing a biodegradable component of an endoluminal device to the endoluminal device until a desired degree of biodegradation has occurred, the method comprising securing the degradable component within a covering of one or more confining materials.

37. A method of enabling an endoluminal device to undergo a change with respect to one or more characteristics over time after implantation without releasing debris greater than a predetermined size into a stream of endoluminal fluid, the method comprising
(a) positioning the device with a first non-biodegradable structural element positioned relative to a second non-biodegradable structural element, the relative position of the first non-biodegradable structural element to the second non-biodegradable structural element controlled by one or more biodegradable structural elements, the device having at least one confining layer positioned between the biodegradable structural element and the stream of endoluminal fluid to prevent debris greater than a predetermined size to pass through the confining layer;
(b) implanting the device in an endoluminal location
(c) inducing the one or more biodegradable structural elements to degrade; and
(d) altering the relative position of the first non-biodegradable structural element to the second non-biodegradable structural element in response to the degradation of the one or more biodegradable structural elements.

38. The method of claim 37, wherein the endoluminal device undergoes a change in size.

39. A method of controlling a biodegradation rate of a first biodegradable element of an endoluminal device relative to a second biodegradable element of the endoluminal device, the method comprising enclosing the first biodegradable element within a first one or more confining members and enclosing the second biodegradable element within a second one or more confining members, the first set of confining members at least one characteristic that is different than the second set of confining members.

40. The method of claim 39, wherein the at least one characteristic is selected from a group consisting of: porosity, permissivity of one or more agents that causes the degradable element to degrade, permissivity of one or more elutants generated by degradation of the degradable element, and receptively to neointimal tissue formation.

41. A method of treating a body lumen having a known first morphology prior to treatment and an expected, different, second morphology after treatment, the method comprising deploying an endoluminal device comprising at least one dynamic structural element adapted to undergo a predictable or controlled change after deployment.

42. An endoluminal prosthesis comprising a stent having one or more biodegradable structural elements at selected locations, the biodegradable structural elements adapted to provide the stent with initial rigidity at the selected locations and to biodegrade in vivo over a period of time to provide a consequent reduction in rigidity at the selected locations.

43. The endoluminal prosthesis of claim 42, wherein the one or more biodegradable structural elements are captured within one or more graft layers adapted to be retained in place upon degradation of the biodegradable element.

44. The endoluminal prosthesis of claim 43, wherein the one or more graft layers have a porosity that selectively permits permeation or non-permeation by degradation products of the biodegradable structural elements, components of surrounding biological fluid, or both.

45. The endoluminal prosthesis of claim 42 further comprising non-biodegradable elements and one or more graft layers adapted to retain the non-biodegradable elements in a position relative to one another following the degradation of the biodegradable elements.

46. The endoluminal prosthesis of claim 43, wherein the one or more graft layers have a porosity that selectively permits permeation or non-permeation by degradation products of the biodegradable structural elements, components of surrounding biological fluid, or both.

47. The endoluminal prosthesis of claim 42 comprising a first non-biodegradable stent at a distal end of the prosthesis, a second non-biodegradable stent at a proximal end of the prosthesis, and one or more biodegradable elements in an intermediate portion of the prosthesis between the distal and proximal stents, and a graft lining or covering extending between the distal stent and the proximal stent.

48. The endoluminal prosthesis of claim 47 wherein a portion of the distal stent extends distally of a distal end of the graft and a portion of the proximal stent extends proximally of a proximal end of the graft.

49. (Cancelled)

50. A medical device comprising a mesh layer comprising one or more polymer encapsulated filaments, each polymer encapsulated filament comprising a non-biodegradable core encapsulated by a biodegradable polymer, wherein the mesh layer is adapted to have a first mesh size before degradation of the biodegradable polymer and a second mesh size after degradation of the biodegradable polymer.

51. The device of claim 50, wherein the mesh layer comprises a radially outward layer of an endoluminal device.

52. The device of claim 50, wherein the second mesh size is large enough to promote intimal growth when the device is deployed.

53. The device of claim 50, further comprising a confining layer radially inward of the mesh layer adapted not to let debris greater than a predetermined size pass through the confining layer.

54. The device of claim 50, wherein the polymer encapsulated filament comprises a co-extruded polymer wire.

55. A medical device comprising a combination of one or more dynamic structural elements and one or more non-dynamic structural elements and having a first structural form immediately after deployment in a body, the device adapted to change in vivo to a second structural form due to a change induced in the one or more dynamic structural elements without releasing debris greater than a predetermined size, said dynamic structural element comprising a biodegradable element contained radially between a first confining member and a second confining member wherein at least one of said confining members is adapted to prevent debris from passing therethrough.
